(19) United States
(12) Patent Application Publication
(10) Pub. No.: US 2016/0030487 A1
(43) Pub. Date: Feb. 4, 2016

(54) METHODS FOR SELECTION OF AGE-APPROPRIATE TISSUES

(71) Applicant: LifeCell Corporation, Branchburg, NJ (US)

(72) Inventors: Nathaniel Bachrach, Clifton, NJ (US); Hui Xu, Plainsboro, NJ (US); Wendell Sun, Warrington, PA (US)

(21) Appl. No.: 14/805,728

(22) Filed: Jul. 22, 2015

Related U.S. Application Data
(60) Provisional application No. 62/030,874, filed on Jul. 30, 2014.

(51) Int. Cl. A61K 35/36

(52) U.S. Cl. CPC A61K 35/36

(57) ABSTRACT

Methods of producing tissue matrices are provided. The methods can comprise selection of collagen-based tissues from animals based on desired mechanical and/or biologic properties relating to the age of a source animal. Furthermore, tissue matrices produced from animals including pigs selected at various ages to control mechanical properties are provided.

This application claims priority under 35 USC §119 to U.S. Provisional Patent Application No. 62/030,874, which was filed on Jul. 30, 2014, and is incorporated by reference in its entirety.

This disclosure relates generally to devices and methods for tissue regeneration and/or repair, and more particularly, to devices incorporating age-appropriate selection of tissue for production of tissue products.

During abdominal surgery, primary incisions are generally closed using sutures and/or other fasteners such as staplers. In some cases, however, it is desirable to incorporate biologic or synthetic materials to assist in wound closure. For example, for some types of hernia operations it is desirable to incorporate biologic or synthetic materials into a surgical site to reinforce the closure and/or provide additional material when there is insufficient or damaged tissue that prevents primary wound closure.

A number of biologic materials are available to assist in tissue regeneration and/or closure of incisions in the abdominal wall or other anatomic locations. For example, one material that is widely used for tissue regeneration is STRATTICE™ (LifeCell Corp, Branchburg, N.J.), which is acellular dermis that has been processed to remove cells and tissue antigens while maintaining the ability to support cellular growth and tissue regeneration and/or remodeling. Although this material is effective for regeneration of tissue and treatment of numerous anatomical sites, it may be further modified to control various mechanical and/or biological properties for certain applications.

Accordingly, the present disclosure provides methods for producing processed animal tissues having mechanical and/or biological properties related to the age of animals from which the animal tissues are derived. Accordingly, the age of the animals that are the source of the tissues used to make the tissue products may be selected based on desired final product specifications.

A method for producing a tissue product is provided. The method can comprise identifying an anatomical site for treatment and identifying a species of animal having a collagen-based tissue for use in treating the anatomical site. In addition, the method can further comprise identifying a desired set of mechanical properties suitable for treating the anatomical site and selecting an animal of the identified species of an age at which a processed collagen-based tissue from the animal will comprise the desire set of mechanical properties. The method can further comprise procuring a collagen-based tissue from the selected animal and processing the collagen-based tissue to produce an acellular tissue matrix from the collagen-based tissue.

In one embodiment, the collagen-based tissue comprises dermis. In some embodiments, the collagen-based tissue is selected fascia, pericardial tissue, dura, umbilical cord tissue, placental tissue, cardiac valve tissue, ligament tissue, tendon tissue, arterial tissue, venous tissue, neural connective tissue, urinary bladder tissue, ureter tissue, and intestinal tissue.

The animal can comprise a pig. Alternatively, the animal is selected from non-human primates, dogs, horses, cats, goats, lambs, monkeys, and cows, or other mammals or non-mammalian animals.

According to various embodiments, the set of properties includes at least one of strength, pliability, suture strength, elasticity, tensile strength, density, stiffness, resistance to enzymatic (e.g., protease) digestion, and compressibility.

Furthermore tissue matrices are provided. The tissue matrices can comprise an acellular dermal matrix derived from dermis of a pig between about 7 and 12 months, and/or a tissue matrix produced according to any of the methods disclosed herein. In addition, methods of treating anatomic sites using the presently disclosed tissue matrices are provided.

In addition, methods for producing tissue products are provided. The methods can comprise identifying an anatomical site for treatment and identifying a species of animal having a collagen-based tissue for use in treating the anatomical site. In addition, the method can further comprise identifying a desired set of mechanical properties suitable for treating the anatomical site and selecting a group of animals of the identified species of an age at which a processed collagen-based tissue from the animal will comprise the desire set of mechanical properties. The method can further comprise procuring collagen-based tissue from the selected animals and processing the collagen-based tissue to produce acellular tissue matrices from the collagen-based tissue. The animals may be selected to be within a desired age range to allow, for example, consistency in material properties, including biological and mechanical properties.

It is to be understood that both the foregoing general description and the following detailed description are exemplary and explanatory only and are not restrictive of the invention, as claimed.

The accompanying drawings, which are incorporated in and constitute a part of this specification, illustrate methods and embodiments of the invention and together with the description, serve to explain the principles of the various aspects of the invention.

### BRIEF DESCRIPTION OF DRAWINGS

FIG. 1 is a bar graph showing variations in porcine acellular dermal tissue density as a function of age.

FIG. 2 illustrates porcine acellular dermal tissue solubility as a function of age.

FIGS. 3A-3B illustrate differential scanning calorimetry (DSC) heat-flow curves for porcine acellular dermal tissue at procured from animals at 6 and 9 months of age, respectively.

FIG. 3C illustrates the peak 2 to peak 1 ratio of DSC curves for porcine acellular dermal tissue procured from animals at 6, 9, and 10 months of age.

FIG. 4 is a bar graph showing variations in porcine acellular dermal tissue sGAG content as a function of age.

FIG. 5 is a bar graph showing variations in porcine acellular dermal tissue elastin content as a function of age.

FIGS. 6A-6C are bar graph showing mechanical properties (maximum load (FIG. 6A), maximum stress (FIG. 6B), and elongation under 16N load (FIG. 6C) of porcine acellular dermal tissue procured from animals at 6 and 9 months of age.

FIG. 7A is a plot of suture retention strengths of porcine acellular dermal tissue procured from animals at 6 and 9 months of age.

FIG. 7B is a plot of burst strengths of porcine acellular dermal tissue derived procured from animals at 6 and 9 months of age.

FIG. 8 illustrates fatigue testing data measured by elongations during cyclic tensile loading of porcine acellular dermal tissue procured from animals at 6 and 9 months of age.

FIGS. 9A-9B illustrate collagenase susceptibility of porcine acellular dermal tissue as a function of age, including free amine content (FIG. 9A) and changes in tissue weight (FIG. 9B) due to collagenase exposure.

FIGS. 10A-10B illustrate maximum stress of porcine acellular dermal tissue after implantation using a rat subcutaneous model with explantation at various times over 42 days of porcine acellular dermal tissue procured from animals at 6 and 9 months of age.

FIG. 10C is a plot of the average measurements for samples at various time points, as derived from the data in FIGS. 10A-10B.

FIGS. 11A-11B are hematoxylin and eosin (H&E) sections of porcine acellular dermal tissue after implantation using a rat subcutaneous and explantation at 42 days for tissue derived from 6 and 9 months old pigs respectively.

### DESCRIPTION OF EXEMPLARY EMBODIMENTS

Reference will now be made in detail to embodiments consistent with the present disclosure, examples of which are illustrated in the accompanying drawings. Wherever possible, the same reference numbers will be used throughout the drawings to refer to the same or like parts.

In this application, the use of the singular includes the plural unless specifically stated otherwise. In this application, the use of “or” means “and/or” unless stated otherwise. Furthermore, the use of the term “including”, as well as other forms, such as “includes” and “included”, is not limiting. Also, terms such as “element” or “component” encompass both elements and components comprising one unit and elements and components that comprise more than one subunit, unless specifically stated otherwise. Also the use of the term “portion” may include part of a moiety or the entire moiety.

The section headings used herein are for organizational purposes only and are not to be construed as limiting the subject matter described. All documents, or portions of documents, cited in this application, including but not limited to patents, patent applications, articles, books, and treatises, are hereby expressly incorporated by reference in their entirety for any purpose.

As used herein, the “age” of an animal refers to the time since birth of the animal after a normal gestational period.

As used herein “tissue product” will refer to any animal tissue that contains extracellular matrix proteins. “Tissue products” can include acellular or partially decellularized tissue matrices, as well as decellularized tissue matrices that have been repopulated with exogenous cells.

As used herein, “tissue matrix” refers to the extracellular matrix, including collagen for a given tissue, wherein the matrix retains the interconnected structural collagen matrix characteristics of the native extracellular matrix from the tissue from which it is derived. “Acellular tissue matrix” refers to a “tissue matrix” from which the native tissue cells have been removed. “Tissue matrices” do not include solubilized or reconstituted collagen materials.

Various human and animal tissues can be used to produce products for treating patients. For example, various tissue products are available for regeneration, repair, augmentation, reinforcement, and/or treatment of human tissues that have been damaged or lost due to various diseases and/or structural damage (e.g., from trauma, surgery, atrophy, and/or long-term wear and degeneration). Such products can include, for example, acellular tissue matrices, tissue allografts or xenografts, and/or reconstituted tissues (i.e., at least partially decellularized tissues that have been seeded with cells to produce viable materials).

For surgical applications, it is often desirable to produce tissue products that have certain mechanical properties. For example, the tissue product, which may include a sheet of material, should possess sufficient strength to withstand stresses during the intended use. Certain tissue products may be used to treat defects (e.g., hernias), to support surrounding tissues or implants (e.g., for breast augmentation and/or reconstruction), or to replace damaged or lost tissue (e.g., after trauma or surgical resection). Whatever the particular use, the tissue product should have sufficient strength, elasticity, and/or other mechanical properties to function until tissue regeneration and/or repair occurs. For abdominal repair, typical mechanical values include, for example, suture retention strength greater than 23N; tensile strength greater than 23N; and burst strength greater than 210N

Furthermore, the tissue product should possess desired biologic properties. For example, regenerative materials such as ALLODERM® and STRATTICE™ (LifeCell Corp, Branchburg, N.J.) should allow ingrowth of recipient cells to allow regeneration of tissue and prevent fibrosis. Furthermore, such materials may be subject to proteolytic degradation and integration with host tissue over time. However, the rate at which the materials are degraded by host enzymes can be important to, for example, control the regenerative process and provide sufficient mechanical support to the treatment site. And the rate of degradation and integration with host tissue can be related to the age of animals that serve as the tissue source.

Some tissue products may be functionally improved by controlling the mechanical properties of the products. For example, a number of acellular tissue matrix products are available, and often, such tissue matrices are in the form of a flexible sheet of material that has substantially uniform mechanical and/or biological properties over its entire surface area. For some indications, however, it may be desirable to control the mechanical and/or biological properties of such tissue matrices. For example, in some embodiments, it may be desirable to strengthen, stiffen, weaken, or make more pliable a tissue product to produce a product having variable mechanical properties. In addition, in some embodiments, it may be desirable to control certain elastic or viscoelastic properties of a tissue matrix, including, for example, the resistance to stretching at low deformation levels. These properties may be controlled by, in part, selected materials based on the age of an animal from which source tissues are obtained. Furthermore, it may be desirable to control the strength, pliability, suture strength, elasticity, tensile strength, density, stiffness, and compressibility of tissue products, as well as the susceptibility to enzymatic digestion, and content of various components such as certain protein (e.g., sGAG and/or elastin).

Accordingly, a method for producing a tissue product is provided. The method can comprise identifying an anatomical site for treatment and identifying a species of animal having a collagen-based tissue for use in treating the anatomical site. In addition, the method can further comprise identifying a desired set of mechanical properties suitable for treating the anatomical site and selecting an animal of the identified species of an age at which a processed collagen-based tissue from the animal will comprise the desire set of mechanical properties. The method can further comprise procuring a collagen-based tissue from the selected animal and processing the collagen-based tissue to produce an acellular tissue matrix from the collagen-based tissue.

The present disclosure also provides methods and devices for treatment of anatomic sites, such as abdominal wall defects as well as defects in other anatomic sites. For example, in one exemplary aspect of the present disclosure, the devices produced according to the disclose methods can be used for treatment of an midline abdominal incision, which is a vertical incision made along the linea alba between the two rectus abdominis muscles of the abdominal wall. In addition, the disclosed devices can be used for treatment of other load-bearing anatomic sites, such as, fascia, tendons, ligaments, bones (e.g., as periosteal replacements), annular disc repair, hernias in any abdominal or torso location, cartilage regeneration, and/or periodontal applications. For abdominal wall treatment or any other load-bearing applications, the disclosed methods and devices and include tissue produced from pigs between 7-12 months old, or between 8-10 months old, or any ranges in between.

For other applications, it may be desirable to use tissue from younger animals. For example, for breast surgery applications (e.g., reconstruction), tissue matrices may be used to provide additional coverage, reinforce tissue, and/or help improve aesthetic outcomes. Generally, however, such procedures benefit from tissue matrices that are more pliable and less stiff than materials used for greater load-bearing applications such as hernia repair. Accordingly, for such applications tissue may be derived from a younger animal, including, for example pigs between, for example, 1-3 months, 2-3 months, 2-4 months, or any ranges in between.

In addition, methods for producing tissue products are provided. The methods can comprise identifying an anatomical site for treatment and identifying a species of animal having a collagen-based tissue for use in treating the anatomical site. In addition, the method can further comprise identifying a desired set of mechanical properties suitable for treating the anatomical site and selecting a group of animals of the identified species of an age at which a processed collagen-based tissue from the animal will comprise the desire set of mechanical properties. The method can further comprise procuring collagen-based tissue from the selected animals and processing the collagen-based tissue to produce acellular tissue matrices from the collagen-based tissue. The animals may be selected to be within a desired age range to allow, for example, consistency in material properties, including biological and mechanical properties. For example, the animals may be selected to from a group such that all animals are within 1 month of age from each of the other animals, within 2 months of age from each of the other animals, or within 3 months of age from each of the other animals.

In one aspect, the devices and methods disclosed herein are used for closing abdominal incisions when primary suture closure is not feasible due to loss of abdominal muscle and/or fascia. In some embodiments, the devices and methods disclosed herein are used for midline incision closure following a surgical procedure. In another aspect, the devices and methods are used as a treatment for ventral or incisional hernia, e.g., for treatment of midline incisional hernia, inguinal hernia repair, or other abdominal defect repair. In various embodiments, the methods and devices of the present disclosure can be used for prophylactic treatment, e.g., to prevent incisional hernia or prevent parastomal hernia. In some embodiments, the methods and devices of the present disclosure can be used to treat preexisting abdominal wall defects or to assist in closure of incisions or hernias where insufficient abdominal tissue is present.

The devices produced according to the disclosed methods can include a number of shapes or configurations. For example, devices suitable for treatment of abdominal incisions and/or hernias can include a pliable sheet of acellular tissue. In certain embodiments, the sheet comprises an acellular tissue matrix that may support revascularization and repopulation of the implanted matrix with the patient's own cells to further strengthen the treatment site and lower the risk of matrix dislodgement. In some embodiments, the acellular tissue matrix includes a dermal matrix. The sheet can be derived from porcine dermis that is processed to remove cells and tissue antigens while maintaining the ability to support cellular growth and tissue regeneration and/or remodeling. In exemplary embodiments, the acellular dermis matrix is derived from skin harvested from the spinal region of porcine, bovine, or other animals.

In various embodiments consistent with the present disclosure, the sheets can be produced from tissue that is xenogenic to a human recipient. Xenogeneic sources can include a variety of different non-human mammals. For example, as noted above, one suitable biologic material for production of the sheets and elongate element is STRATTICE™, which is a porcine-derived tissue matrix. However, other xenograft sources can be used.

Xenogenic tissues can be processed to remove antigens known to elicit an immune response in the recipient. For example, various decellularization processes or enzyme treatments are known that allow removal of cellular and/or extracellular antigens that may be immunogenic. Further, in various embodiments, the tissues can be derived from animals that are genetically modified or altered to have diminished expression of antigens known to be immunogenic in humans. For example, in one such embodiment, the tissues are harvested from an α1,3-galactosyltransferase (α1,3GT) deficient pig or other animal to prevent hyperacute rejection of the implant by the recipient. Different methods of producing α1,3GT deficient pigs have been previously described in Dai, Y. et al., “Targeted disruption of the α1,3-galactosyltransferase gene in cloned pigs,” Nat. Biotechnology 20, 251-255 (2002), and Phelps, C. J. et al., “Production of α1,3-galactosyltransferase-deficient pigs,” Science 299, 411-413 (2003), which are incorporated herein by reference.

### Decellularized Tissue Products

In various embodiments, a tissue product can comprise an intact or decellularized tissue from an animal. In some embodiments, the tissue can be partially or completely decellularized but retains at least some components of the extracellular matrix into which native cells from tissue surrounding an implanted tissue product can migrate and proliferate, thereby enhancing the speed or overall level of repair, regeneration, healing, or treatment of native tissue. Decellularization can be done before, at the same time, and/or after other processing steps such as enzymatic exposure to remove certain antigenic components and/or to control the mechanical or biologic properties of the material.

The extracellular matrix within the decellularized elements of a tissue product may consist of collagen, elastin, and/or other fibers, as well as proteoglycans, polysaccharides and/or growth factors. In some embodiments, a decellularized tissue matrix may retain some or all of the extracellular matrix components that are found naturally in a tissue prior to decellularization, or various undesirable components may be removed by chemical, enzymatic or genetic means. In general, the acellular matrix provides a structural network on which native tissue and vasculature can migrate, grow, and proliferate. The exact structural components of the extracellular matrix will depend on the tissue selected and the processes used to prepare the acellular tissue.

In some embodiments, a tissue product can be derived from any tissue that is suitable for decellularization and subsequent implantation. Exemplary tissues include, but are not limited to, bone, skin, adipose tissue, dermis, intestine, urinary bladder, tendon, ligament, muscle, fascia, neurologic tissue, vessel, liver, heart, lung, kidney, cartilage, and/or any other suitable tissue. In certain embodiments, the tissue product can include a decellularized soft tissue. For example, the tissue product can include partially or completely decellularized dermis. In other embodiments, the tissue product can comprise partially or completely decellularized small intestine submucosa.

Exemplary methods for decellularizing tissue are disclosed in U.S. Pat. No. 6,933,326 and U.S. Patent Application 2010/0272782, which are hereby incorporated by reference in their entirety. In various embodiments, the general steps involved in the production of a partially or completely decellularized tissue matrix include harvesting tissue from a donor source and removing cells under conditions that preserve biological and structural function. In certain embodiments, the harvested tissue can be washed to remove any residual cryoprotectants and/or other contaminants. Solutions used for washing can be any physiologically-compatible solution. Examples of suitable wash solutions include distilled water, phosphate buffered saline (PBS), or any other biocompatible saline solution.

In certain embodiments, the decellularization process includes chemical treatment to stabilize the harvested tissue so as to avoid biochemical and structural degradation before, during, or after cell removal. In various embodiments, the stabilizing solution arrests and prevents osmotic, hypoxic, autolytic, and/or proteolytic degradation; protects against microbial contamination; and/or reduces mechanical damage that can occur during decellularization of tissues that contain, for example, smooth muscle components (e.g., blood vessels). The stabilizing solution may contain an appropriate buffer, one or more antioxidants, one or more oncotic agents, one or more antibiotics, one or more protease inhibitors, and/or one or more smooth muscle relaxants.

In various embodiments, the tissue is then placed in a decellularization solution to remove some or all viable cells (e.g., epithelial cells, endothelial cells, smooth muscle cells, and fibroblasts, etc.) from the extracellular matrix without damaging the biological and/or structural integrity of the extracellular matrix. The decellularization solution may contain an appropriate buffer, salt, an antibiotic, one or more detergents (e.g., TRITON X-100™, sodium dodecyl sulfate, sodium deoxycholate, polyoxyethylene (20) sorbitan mono-oleate, etc.), one or more agents to prevent cross-linking, one or more protease inhibitors, and/or one or more enzymes. In some embodiments, the decellularization solution comprises 0.1%, 0.2%, 0.3%, 0.4%, 0.5%, 1.0%, 1.5%, 2.0%, 2.5%, 3.0%, 3.5%, 4.0%, 4.5%, or 5.0% (or any percentage in between) of TRITON X-100™ and, optionally, 10 mM, 15 mM, 20 mM, 25 mM, 30 mM, 35 mM, 40 mM, 45 mM, or 50 mM EDTA (ethylenediaminetetraacetic acid) (or any concentration in between). In some embodiments, the tissue is incubated in the decellularization solution at 25, 30, 31, 32, 33, 34, 35, 36, 37, 38, 39, 40, 41, or 42° C. (or any temperature in between), and optionally with gentle shaking at 10, 20, 30, 40, 50, 60, 70, 80, 90, 100, 110, 120, 130, 140, or 150 rpm (or any rpm in between). The incubation can be for 1, 2, 3, 4, 5, 6, 7, 8, 9, 10, 11, 12, 15, 20, 24, 36, or 48 hours (or any time in between). The length of time or concentration of detergent can be adjusted in order to partially or more fully decellularize the tissue. In certain embodiments, additional detergents may be used to remove fat from the tissue sample. For example, in some embodiments, 1, 2, 3, 4, or 5% sodium deoxycholate (or any percentage in between) is added to the decellularization solution in order to remove fat from the tissue.

In certain embodiments, decellularization completely or substantially removes all cells normally present in the tissue from which the tissue product is derived. As used herein, “substantially free of all cells” means that the tissue product contains less than 20%, 10%, 5%, 1%, 0.1%, 0.01%, 0.001%, or 0.0001% (or any percentage in between) of the cells that normally grow within the acellular matrix of the tissue prior to decellularization.

In some embodiments, after decellularization, the tissue is washed thoroughly. Any physiologically compatible solutions can be used for washing. Examples of suitable wash solutions include distilled water, phosphate buffered saline (PBS), or any other biocompatible saline solution. In certain embodiments, the decellularized tissue is then treated (e.g., overnight at room temperature) with a deoxyribonuclease (DNase) solution to remove xenogenic DNA that could induce an immune response upon implantation. In some embodiments, the tissue sample is treated with a DNase solution prepared in DNase buffer (20 mM HEPES (4-(2-hydroxyethyl)-1-piperazineethanesulfonic acid), 20 mM CaCl₂ and 20 mM MgCl₂). Optionally, an antibiotic solution (e.g., Gentamicin) may be added to the DNase solution. Any suitable DNase buffer can be used, as long as the buffer provides for suitable DNase activity.

While an acellular tissue matrix may be made from one or more individuals of the same species as the recipient of the acellular tissue matrix graft, this is not necessarily the case. Thus, for example, an acellular tissue matrix may be made from porcine tissue and implanted in a human patient. Species that can serve as recipients of acellular tissue matrix and donors of tissues or organs for the production of the acellular tissue matrix include, without limitation, mammals, such as humans, nonhuman primates (e.g., monkeys, baboons, or chimpanzees), pigs, cows, horses, goats, sheep, dogs, cats, rabbits, guinea pigs, gerbils, hamsters, rats, or mice. Elimination of the α-gal epitopes from the collagen-containing material may diminish the immune response against the collagen-containing material. The α-gal epitope is expressed in non-primate mammals and in New World monkeys (monkeys of South America) as well as on macromolecules such as proteoglycans of the extracellular components. U. Galili et al., J. Biol. Chem. 263: 17755 (1988). This epitope is absent in Old World primates (monkeys of Asia and Africa and apes) and humans, however. Id. Anti-gal antibodies are produced in humans and primates as a result of an immune response to α-gal epitope carbohydrate structures on gastrointestinal bacteria. U. Galili et al., Infect. Immun. 56: 1730 (1988); R. M. Hamadeh et al., J. Clin. Invest. 89: 1223 (1992). Since non-primate mammals (e.g., pigs) produce α-gal epitopes and these epitopes are still present within the tissue matrix after decellularization processing, xenotransplantation of collagen-containing material from these mammals into primates can result in an immune response because of primate anti-Gal binding to these epitopes on the collagen-containing material. The interaction of naturally occurring anti-α-gal antibody with these epitopes on acellular matrices has been reported to cause a significant inflammatory response in primate despite no immediate rejection of the implant. See Galili, U. et al., “Porcine and bovine cartilage transplants in cynomolgus monkey,” Transplantation 63, 646, 1997; Stone, K R et al., “Porcine and bovine cartilage transplants in cynomolgus monkey: I. A model for chronic xenograft rejection,” Transplantation 63, 640, 1997; and Hui Xu et al., “A Porcine-Derived Acellular Dermal Scaffold That Supports Soft Tissue Regeneration: Removal of Terminal Galactose-α-(1,3)-Galactose and Retention of Matrix Structure,” Tissue Eng. Part A. July 2009, 15(7): 1807-1819. Furthermore, xenotransplantation results in major activation of the immune system to produce increased amounts of high affinity anti-gal antibodies. Accordingly, in some embodiments, when animals that produce α-gal epitopes are used as the tissue source, the substantial elimination of α-gal epitopes from extracellular components of the collagen-containing material can diminish the immune response against the collagen-containing material associated with anti-gal antibody binding to α-gal epitopes.

To remove α-gal epitopes, after washing the tissue thoroughly with saline to remove the DNase solution, the tissue sample may be subjected to one or more enzymatic treatments to remove certain immunogenic antigens, if present in the sample. In some embodiments, the tissue sample may be treated with an α-galactosidase enzyme to eliminate α-gal epitopes if present in the tissue. In some embodiments, the tissue sample is treated with α-galactosidase at a concentration of 300 U/L prepared in 100 mM phosphate buffer at pH 6.0. In other embodiments, the concentration of α-galactosidase is increased to 400 U/L for adequate removal of the α-gal epitopes from the harvested tissue. Any suitable enzyme concentration and buffer can be used as long as sufficient removal of antigens is achieved.

Alternatively, rather than treating the tissue with enzymes, animals that have been genetically modified to lack one or more antigenic epitopes may be selected as the tissue source. For example, animals (e.g., pigs) that have been genetically engineered to lack the terminal α-galactose moiety can be selected as the tissue source. For descriptions of appropriate animals see co-pending U.S. application Ser. No. 10/896,594 and U.S. Pat. No. 6,166,288, the disclosures of which are incorporated herein by reference in their entirety. In addition, certain exemplary methods of processing tissues to produce acellular matrices with or without reduced amounts of or lacking alpha-1,3-galactose moieties, are described in Xu, Hui. et al., “A Porcine-Derived Acellular Dermal Scaffold that Supports Soft Tissue Regeneration: Removal of Terminal Galactose-α-(1,3)-Galactose and Retention of Matrix Structure,” Tissue Engineering, Vol. 15, 1-13 (2009), which is incorporated by reference in its entirety.

In certain embodiments, after decellularization of a tissue in a tissue product, histocompatible/viable cells may optionally be seeded in the acellular tissue matrix. In some embodiments, histocompatible viable cells may be added to the matrices by standard in vitro cell co-culturing techniques prior to transplantation, or by in vivo repopulation following transplantation. In vivo repopulation can be by the migration of native cells from surrounding tissue into the tissue matrix or by infusing or injecting histocompatible cells obtained from the recipient or from another donor into the tissue matrix in situ. Various cell types can be used, including stem cells such as embryonic stem cells and/or adult stem cells. Any other viable cells that are histocompatible with the patient in which they are being implanted can also be used. In some embodiments, the histocompatible cells are mammalian cells. Such cells can promote native tissue migration, proliferation, and/or vascularization. In certain embodiments, the cells can be directly applied to the tissue matrix just before or after implantation.

In some embodiments, a tissue product can be treated to reduce a bioburden (i.e., to reduce the number of microorganisms growing on the tissue). In some embodiments, the tissue product is treated such that it lacks substantially all bioburden (i.e., the tissue product is aseptic or sterile). As used herein, “substantially all bioburden” means that the concentration of microorganisms growing on the tissue product is less than 1%, 0.1%, 0.01%, 0.001%, or 0.0001% of that growing prior to bioburden treatment, or any percentage in between. Suitable bioburden reduction methods are known to one of skill in the art and may include exposing the tissue product to radiation. Irradiation may reduce or substantially eliminate bioburden. In some embodiments, an absorbed dose of 15-17 kGy of E-beam radiation is delivered in order to reduce or substantially eliminate bioburden. In various embodiments, the amount of radiation to which the tissue product is exposed can be between about 5 Gy and 50 kGy. Suitable forms of radiation can include gamma radiation, e-beam radiation, and X-ray radiation. Other irradiation methods are described in U.S. Application 2010/0272782, the disclosure of which is hereby incorporated by reference in its entirety.

In some embodiments, one or more additional agents can be added to the tissue product. In some embodiments, the additional agent can comprise an anti-inflammatory agent, an analgesic, or any other desired therapeutic or beneficial agent. In certain embodiments, the additional agent can comprise at least one added growth or signaling factor (e.g., a cell growth factor, an angiogenic factor, a differentiation factor, a cytokine, a hormone, and/or a chemokine). These additional agents can promote native tissue migration, proliferation, and/or vascularization. In some embodiments, the growth or signaling factor is encoded by a nucleic acid sequence contained within an expression vector. Preferably, the expression vector is in one or more of the viable cells that can be added, optionally, to the tissue product. As used herein, the term “expression vector” refers to any nucleic acid construct that is capable of being taken up by a cell, contains a nucleic acid sequence encoding a desired protein, and contains the other necessary nucleic acid sequences (e.g. promoters, enhancers, termination codon, etc.) to ensure at least minimal expression of the desired protein by the cell.

The tissue products, as described above, may be provided packaged, frozen, freeze-dried, and/or dehydrated. In certain embodiments, the packaged tissue products are sterile. For example, a kit can comprise a hydrated, frozen, freeze-dried, and/or dehydrated tissue product and instructions for preparing and/or using the tissue products.

### Examples

### Study of Porcine Acellular Dermal Materials as a Function of Animal Age

Various mechanical, structural, and biologic properties of porcine acelllular dermal materials were studied to determine how those properties varied with pig age. For each of the studies, porcine dermis was obtained at various time points (e.g., 6, 9, or 10 months after birth), and the dermis was processed to remove cellular components while retaining the collagen and proteoglycan structure of the dermis. The specific process used is discussed generally in Connor, J. et al., “Retention of structural and biochemical integrity in a biological mesh supports tissue remodeling in a primate abdominal wall model,” Regenerative Medicine (2009) Vol. 4(2):185-195.

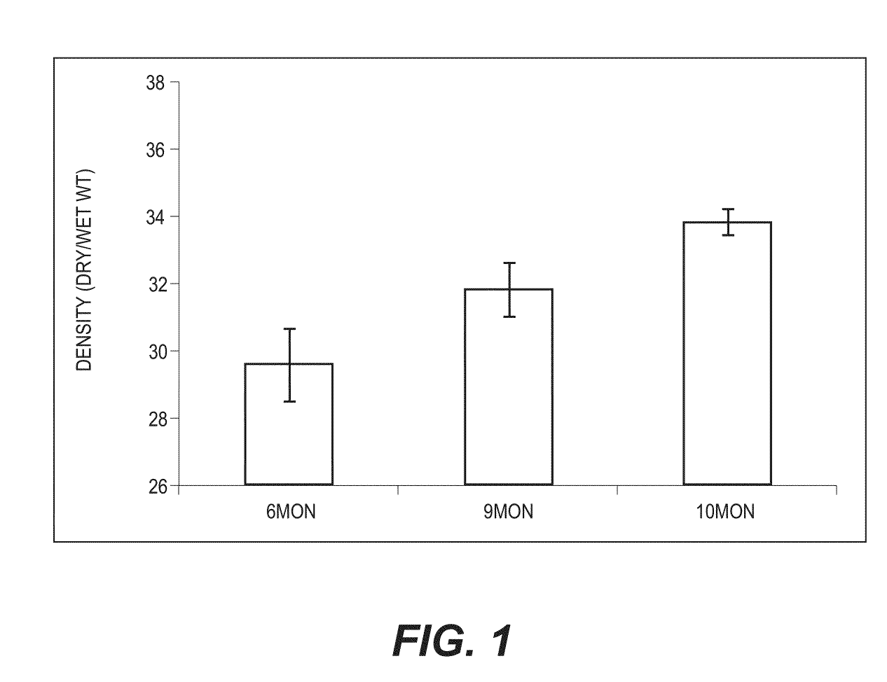

FIG. 1 is a bar graph showing variations in porcine acellular dermal tissue density as a function of age. The changes in tissue density due to an increase in age. To conduct this analysis, porcine acellular dermal matrix (PADM) tissue was cut to 8 mm biopsy punch size and excess fluid was removed with paper towels. The wet tissue weight was determined and the tissue was freeze dried overnight to determine the dry weight of the tissue. The density (percent ratio to initial wet weight) of 6, 9, and 10 month old porcine acellular dermal matrix (PADM) was determined by the ratio of dry and wet tissue weight. Both 9 and 10 month old PADM demonstrated a positive relationship between tissue density and pig age, i.e., tissue density increase with animal age, which suggests that PADM, when implanted, will result in slower tissue turnover and increased tissue stiffness.

### Tissue Solubility

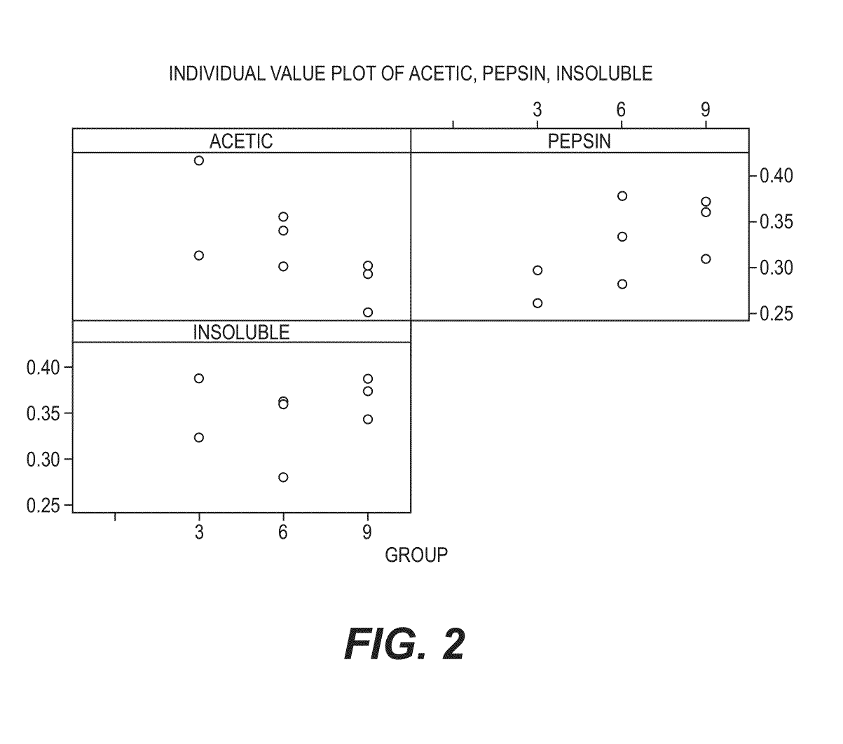

FIGS. 2A-C illustrate porcine acellular dermal tissue solubility as a function of age. To measure tissue solubility, PADM tissue was sequentially extracted with acetic acid and pepsin. Hydroxyproline was measured in extracts and on the remaining insoluble material. Younger hides demonstrated more acetic acid soluble collagen than older hides, while the opposite trend was demonstrated for pepsin soluble collagen. These findings suggest that collagen from older hides is more mature and more durable than collagen from younger hides.

### Differential Scanning Calorimetry

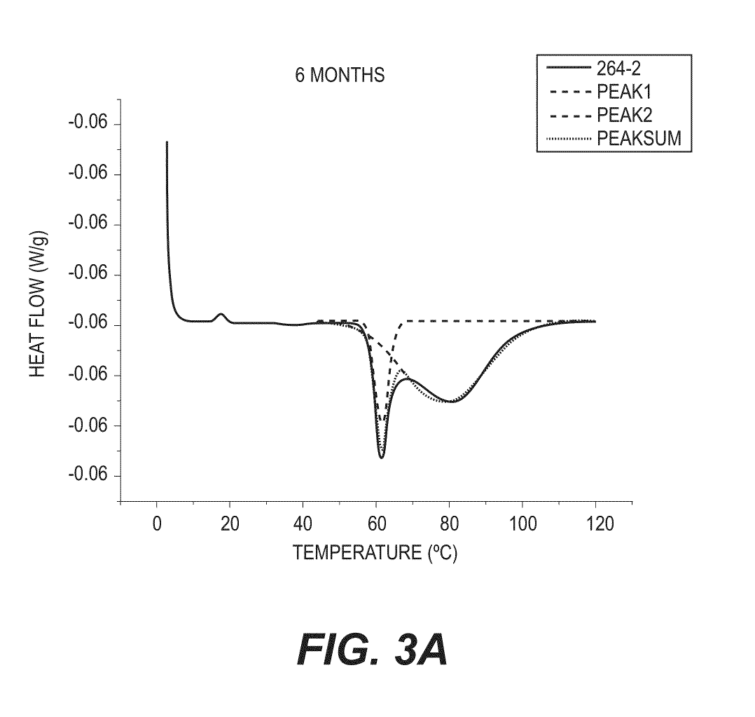

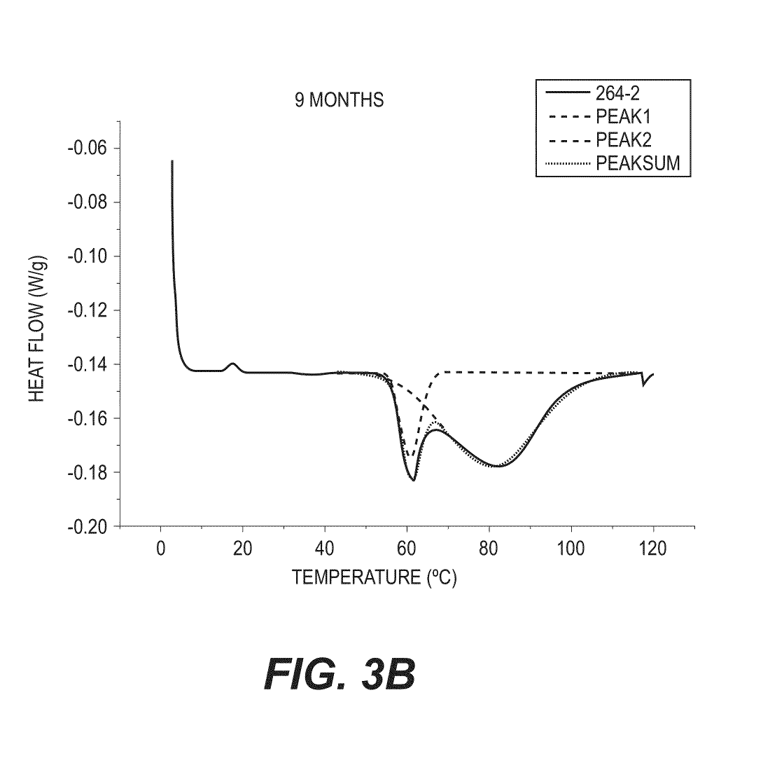

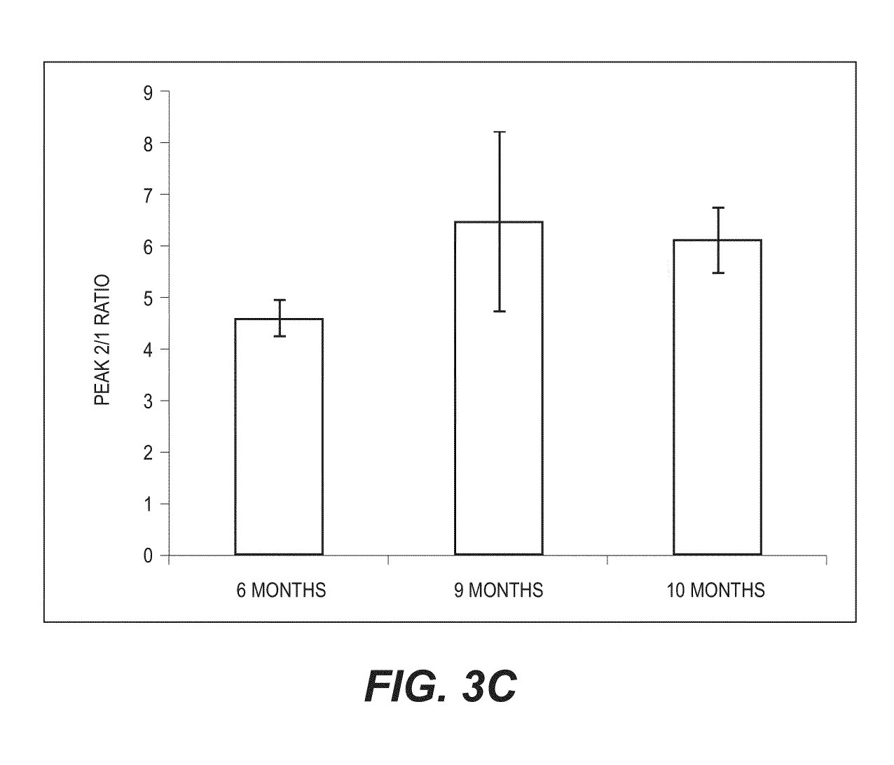

Changes in thermal melting behavior of PADM was determined with Differential Scanning calorimetry (DSC) (Q2000, TA Instruments). The thermal behavior of the tissue was evaluated between 0° C. to 120° C. at an increasing rate of 3° C./minute. FIGS. 3A-3B illustrate differential scanning calorimetry (DSC) heat-flow curves for porcine acellular dermal tissue at 6 and 9 months respectively. FIG. 3C illustrates the peak 2 to peak 1 ratio of DSC curves for 6, 9, and 10 month old porcine acellular dermal tissue. DSC results demonstrate no significant changes in the onset temperature of the tissue (peak 1) but significant difference in the magnitude of the second peak (FIGS. 3A and B).

The DSC results was further fitting with two different peaks to determine a 2nd vs. 1st peak melting energy ratio (FIG. 3C). This demonstrates that the 9 and 10 month old PADM was more thermally stable, potentially due to potential collagen biochemical changes and cross-linking, which likely correlates with slower degradation in vivo.

sGAG Content and Elastin Content

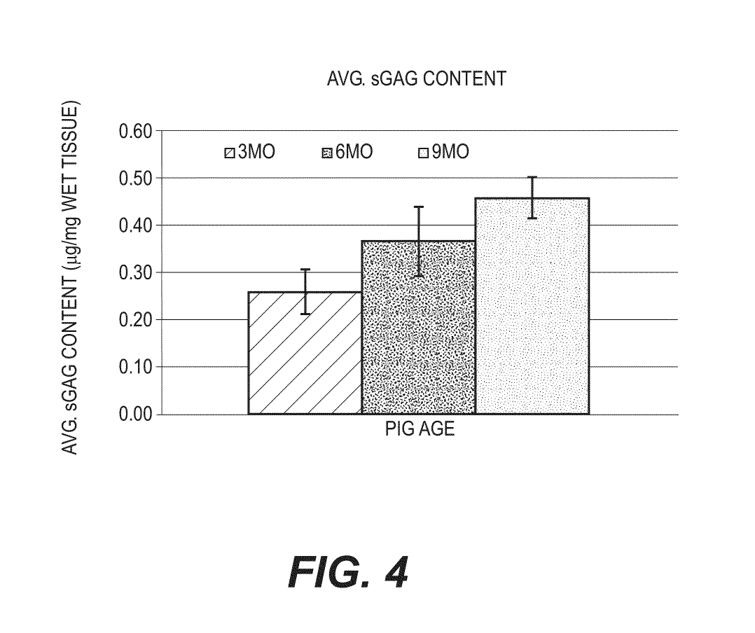

The presence of GAGs in PADM from approximately 3, 6 and 9 month old hides was evaluated using a commercially available sulfated glycosaminoglycan assay kit by BIOCOLOR. FIG. 4 is a bar graph showing variations in porcine acellular dermal tissue sGAG content as a function of age. The results showed an increase of sGAG content with age.

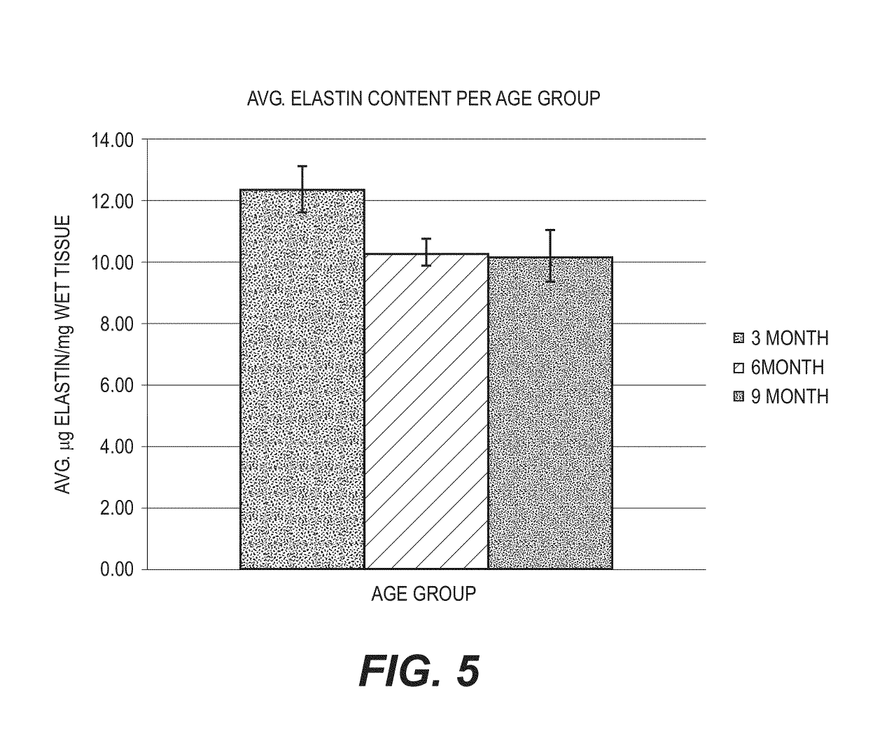

The presence of elastin in post processed pADM from approximately 3, 6, and 9 month old hides was evaluated by a commercially available elastin assay kit from BIOCOLOR. FIG. 5 is a bar graph showing variations in porcine acellular dermal tissue elastin content as a function of age. The results showed a decrease of elastin content in 6 and 9 month hides when compared to 3-month hide.

### Mechanical Testing

Mechanical testing was performed to measure maximum load, maximum stress, tissue elongation under a 16N load, burst strength and fatigue properties.

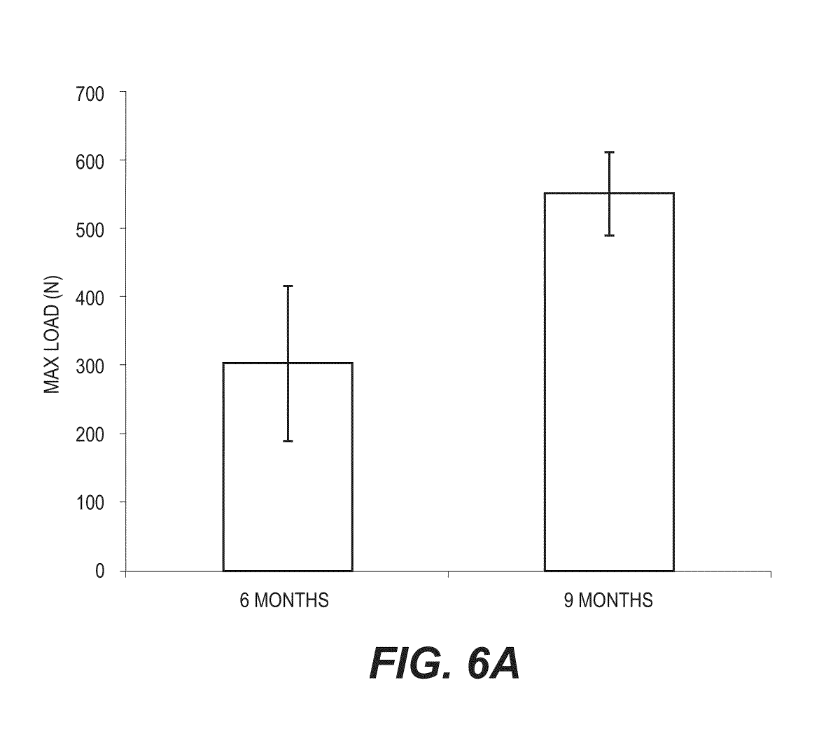

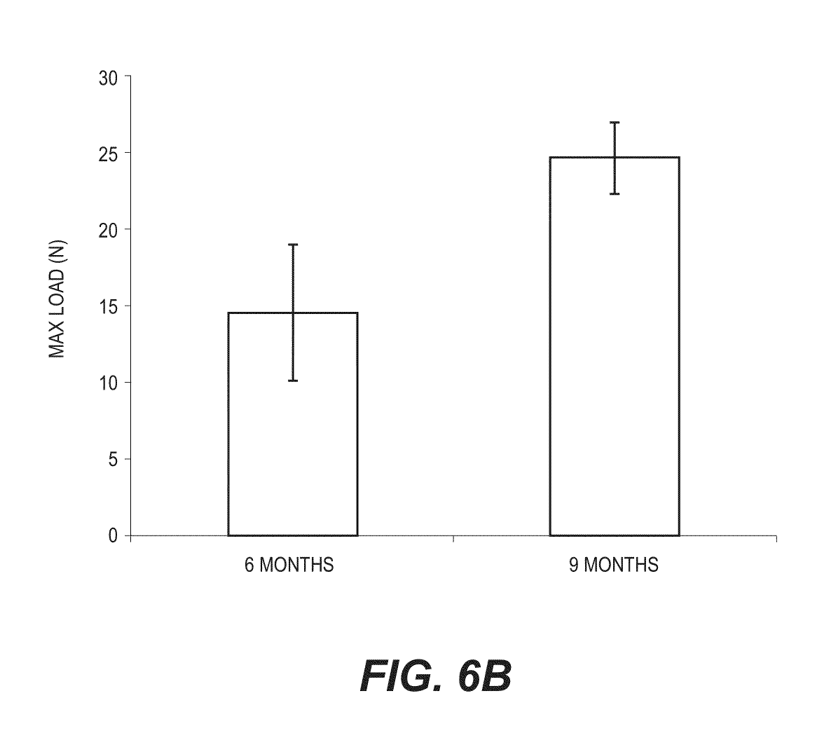

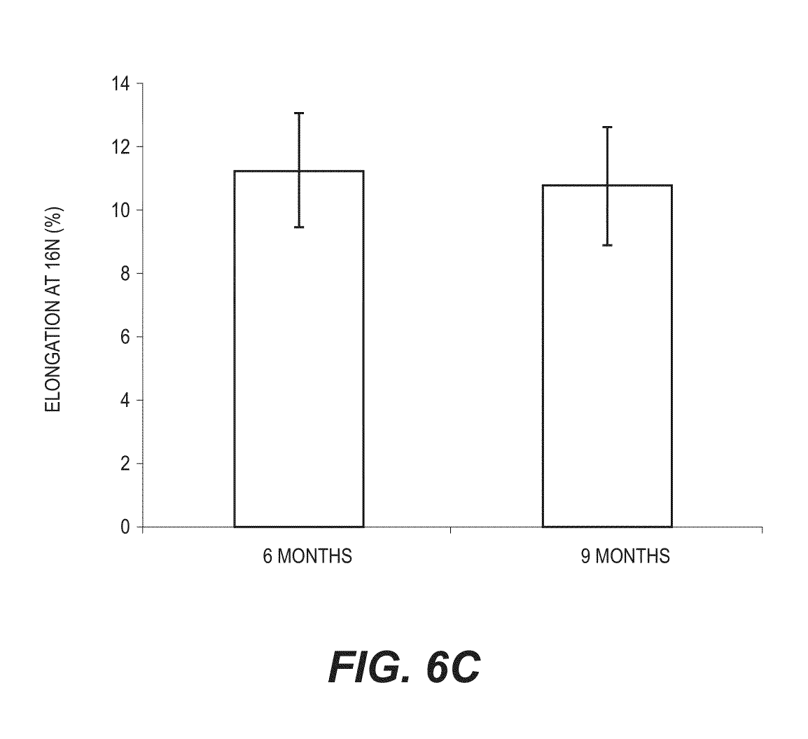

The mechanical properties of PADM at 6 and 9 month old was determined using a tension tester. FIGS. 6A-6C are bar graph showing mechanical properties (maximum load (FIG. 6A), maximum stress (FIG. 6B), and elongation under 16N load (FIG. 6C) of porcine acellular dermal tissue derived from 6 and 9 months old pigs. FIGS. 6A and B demonstrate illustrate an increasing maximum load and maximum stress with increasing animal age, while FIG. 6C shows that the elongation of 9 month old tissue at low loads (16N) is not significantly different from 6 month old tissue.

The suture retention strength of pADM from 6 and 9 month old hides was evaluated by suture pull through test using a tension tester (5900 TESTING SYSTEM, INSTRON), and the burst strength of pADM from 6 and 9 month old hides was evaluated by burst testing using the same system. For suture retention, 2 cm×3 cm samples were pulled at a controlled extension rate of 750 mm/min until failure, and for burst strength, 8 cm×8 cm samples were loaded on a ring clamp and tested with a steel ball speed of 305 mm/min.

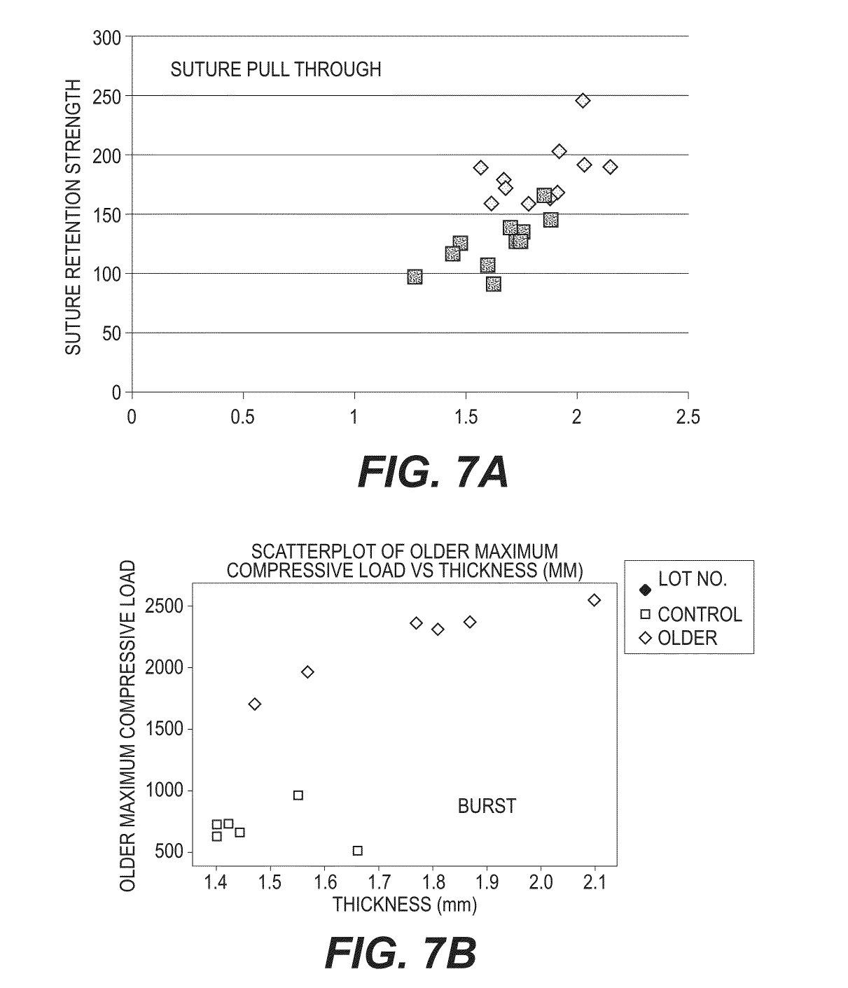

FIG. 7A is a plot of suture retention strengths for porcine acellular dermal tissue derived from 6 and 9 months old pigs, and FIG. 7B is a plot of burst strengths for porcine acellular dermal tissue derived from 6 and 9 months old pigs. As shown, both suture retention strength and burst strength increase with animal age.

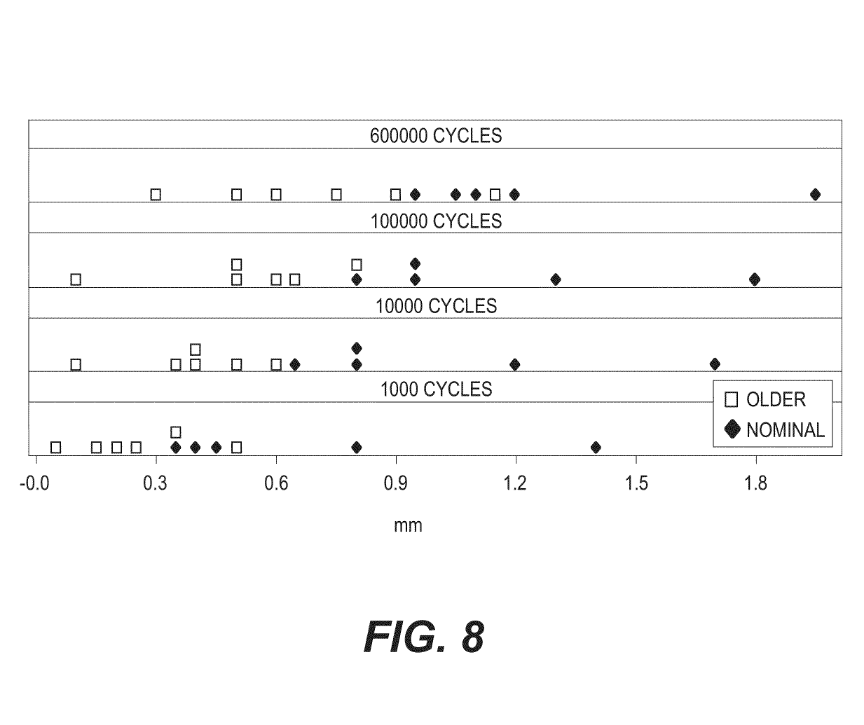

Finally, fatigue testing of PADM from 6 and 9 month old hides was performed. Testing was conducted up to 600,000 cycles on a dynamic tension tester (8874, INSTROn) with an applied load of 10N/cm at 6 Hz. FIG. 8 illustrates fatigue testing data measured by elongations during cyclic tensile loading for porcine acellular dermal tissue derived from 6 and 9 months old pigs. 9 month old hides demonstrated less elongation at all cycles indicating lower fatiguability for PADM produced from older animals.

### Collagenase Susceptibility

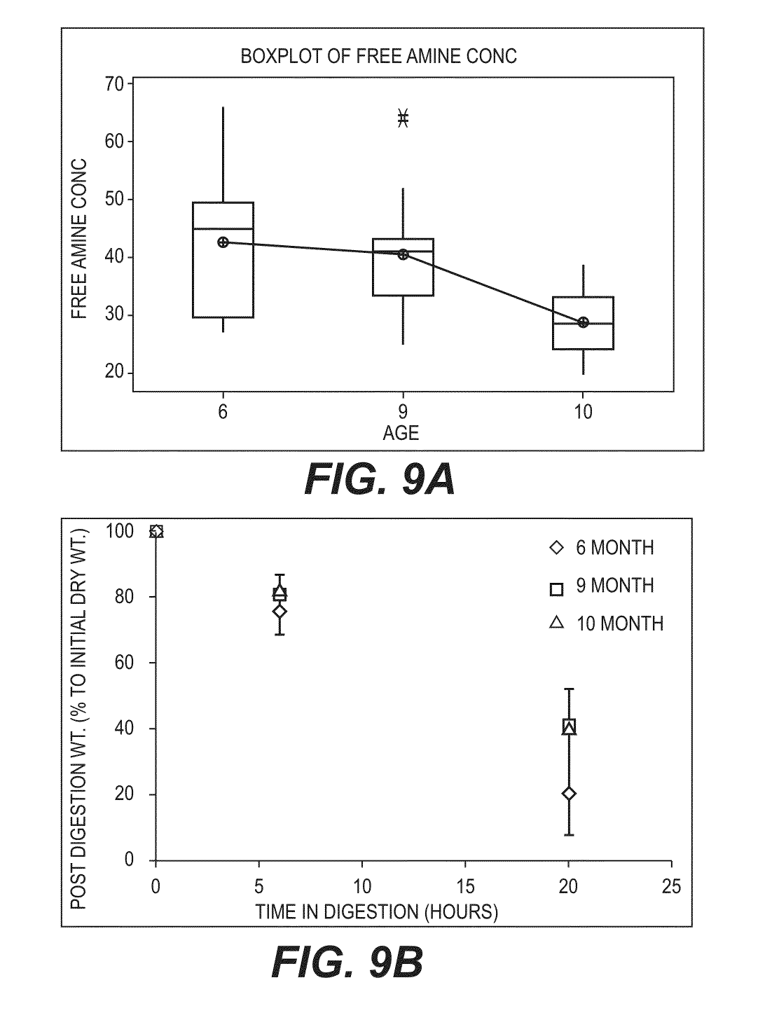

PADM susceptibility to collagenase digestion was studied by A) quantification of released free amine and B) changes in tissue weight as a function of collagenase digestion. FIGS. 9A-9B illustrate collagenase susceptibility of porcine acellular dermal tissue as a function of age, including free amine content (FIG. 9A) and changes in tissue weight (FIG. 9B) due to collagenase exposure.

To study release of free amines, the released free amine content in the supernatant was measured via the reaction chemistry between picryl sulfonic acid and the solution free amine content. In this assay, 10 month old PADM demonstrated greater resistance to collagenase digestion due to the reduced level of detectable free amine content in the supernatant.

Further, the collagenase resistance of the PADM was determined by the mass loss of the PADM matrix incubated in collagenase solution. As shown in FIG. 9B, at 20 hours post digestion, the 9 and 10 month old PADM had a greater amount of dry tissue matrix left compared to the 6 month old PADM. Overall, the results demonstrate increased collagenase resistance with older animal tissue, which may correlate with a slower rate of tissue turnover in vivo.

### In Vivo Studies: Changes in Mechanical Properties and Histology of Implanted Tissues as a Function of Age

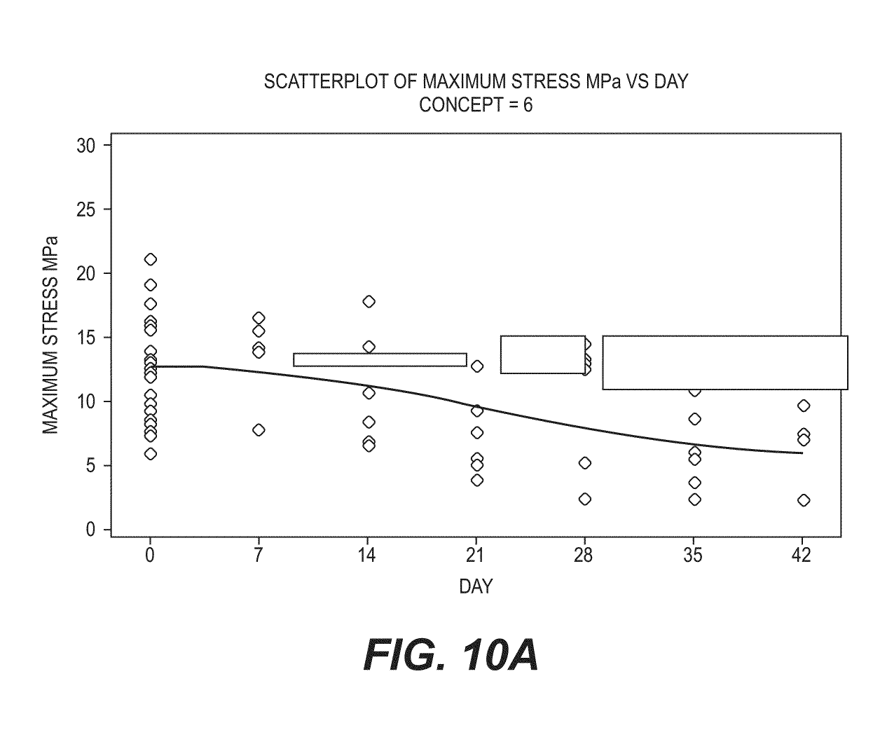

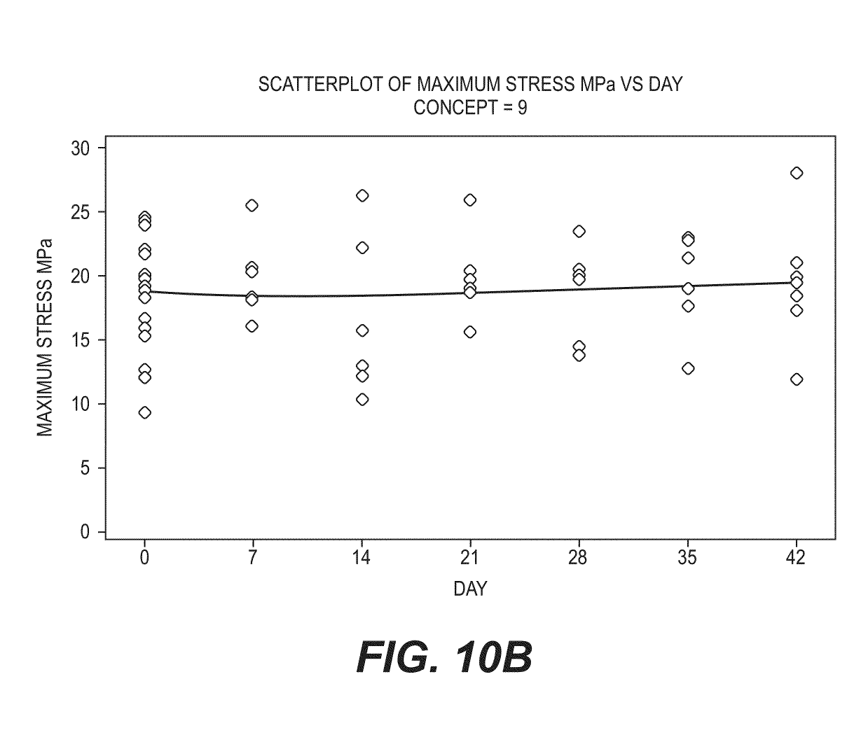

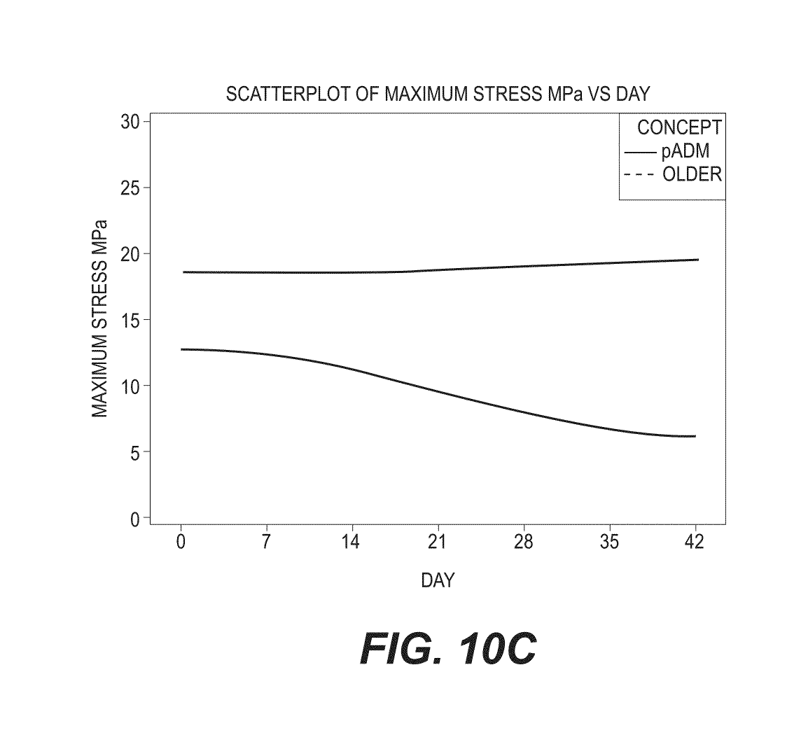

Changes in mechanical properties of PADM after implantation were evaluated in a rat subcutaneous model. PADM tissues (2×6 cm) from 6 and 9 months hides were implanted in rats and explanted at designated dates. The mechanical properties of PADM explants were determined using a tension tester (5900 TESTING SYSTEM, INSTRON). FIGS. 10A-10B illustrate maximum stress of porcine acellular dermal tissue after implantation using a rat subcutaneous model with explantation at various times over 42 days for tissue derived from 6 and 9 months old pigs respectively. FIG. 10C was plotted using the average of maximum stress of the explants from FIGS. 10A and 10B. The data demonstrates that PADM from hides of 9 month old pigs is more durable than that from 6 month old pigs.

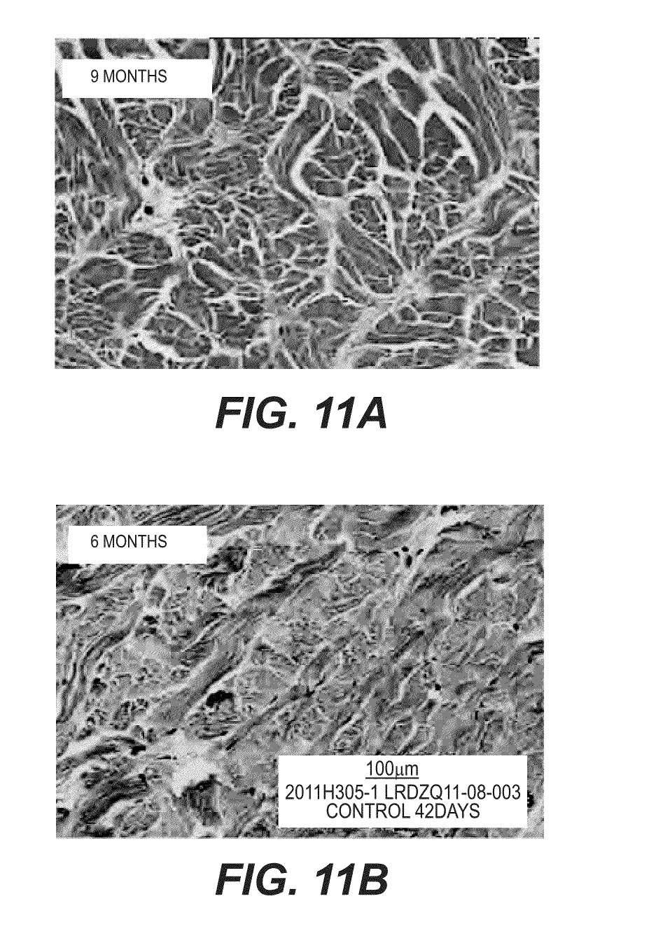

Host biological response to PADM of 6 and 10 months hide was evaluated in a rat subcutaneous model at 14 and 28 days. FIGS. 11A-11B are hematoxylin and eosin (H&E) sections of porcine acellular dermal tissue after implantation using a rat subcutaneous and explantation at 42 days for tissue derived from 6 and 9 months old pigs respectively. The data shows presence of host cells and new vascular structures, absence of encapsulation and no significant inflammation in any PADM tested. Therefore, PADM from hides of 10 month old pigs shows a similar biological response to that from 6 months old pigs.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A method for producing a tissue product, comprising:
identifying an anatomical site for treatment;
identifying a species of animal having a collagen-based tissue for use in treating the anatomical site;
identifying a desired set of mechanical properties suitable for treating the anatomical site;
selecting an animal of the identified species of an age at which a processed collagen-based tissue from the animal will comprise the desire set of mechanical properties;
procuring a collagen-based tissue from the selected animal; and
processing the collagen-based tissue to produce an acellular tissue matrix from the collagen-based tissue.

2. The method of claim 1, wherein the collagen-based tissue comprises dermis.

3. The method of claim 1, wherein the collagen-based tissue is selected fascia, pericardial tissue, dura, umbilical cord tissue, placental tissue, cardiac valve tissue, ligament tissue, tendon tissue, arterial tissue, venous tissue, neural connective tissue, urinary bladder tissue, ureter tissue, and intestinal tissue.

4. The method of claim 1, wherein the animal comprises a pig.

5. The method of claim 1, wherein the animal is selected from non-human primates, dogs, horses, cows.

6. The method of claim 1, wherein the set of mechanical properties includes at least one of strength, pliability, suture strength, elasticity, tensile strength, density, stiffness, and compressibility.

7. The method of claim 1, wherein the animal is further selected at an age at which the selected tissue has a desired degree of resistance to enzymatic digestion.

8. The method of claim 1, further comprising treating the tissue to removal alpha-1,3-galactose epitopes.

9. The method of claim 1, wherein the acellular tissue matrix comprises a pliable sheet.

10. The method of claim 1, wherein the selected animal is a pig between about 7 and 12 months old and the collagen-based tissue comprises dermis.

11. The method of claim 1, wherein the selected animal is a pig between about 8 and 10 months old and the collagen-based tissue comprises dermis.

12. A tissue matrix, comprising:
an acellular dermal matrix derived from dermis of a pig between about 7 and 12 months.

13. The tissue matrix of claim 12, wherein the dermal matrix is derived from a pig between about 8 and 10 months old.

14. The tissue matrix of claim 12, wherein the tissue matrix has a strength sufficient for use as an abdominal wall treatment device.

15. The tissue matrix of claim 12, wherein the tissue matrix lacks alpha-1,3-galactose epitopes.

16. The tissue matrix of claim 15, wherein the tissue matrix has been treated to remove alpha-1,3-galactose epitopes.

17. The tissue matrix of claim 12, wherein the tissue matrix comprises a pliable sheet.

18. An acellular tissue matrix produced according to the methods of claim 1.

19. A method of treating an anatomic site, comprising:
selecting an anatomic site having a defect;
selecting an acellular dermal matrix derived from dermis of a pig between about 7 and 12 months; and
securing the acellular dermal matrix in or on the anatomic site to treat the defect.

20. The method of claim 19, wherein the dermal matrix is derived from a pig between about 8 and 10 months old.

21. The method of claim 19, wherein the anatomic site comprises a load-bearing tissue.

22. The method of claim 21, wherein the anatomic site comprises a portion of an abdominal wall.

23. The method of claim 21, wherein the anatomic site comprises a midline abdominal incision.

24. The method of claim 21, wherein the anatomic site comprises an abdominal hernia.

25. The method of claim 19, wherein the acellular dermal matrix lacks alpha-1,3-galactose epitopes.

26. The method of claim 25, wherein the acellular dermal matrix has been treated to remove alpha-1,3-galactose epitopes.

27. The method of claim 19, wherein the acellular dermal matrix comprises a pliable sheet.
